(19) United States
(12) Patent Application Publication
(10) Pub. No.: US 2023/0069586 A1
(43) Pub. Date: Mar. 2, 2023

(54) METHODS OF USING ANDROGRAPHOLIDE, ORIDONIN AND ISOLIQUIRITIGENIN AND DERIVATIVES THEREOF

(71) Applicant: THE REGENTS OF THE UNIVERSITY OF CALIFORNIA, Oakland, CA (US)

(72) Inventors: Brian C. LEONARD, Davis, CA (US); Christopher J. Murphy, Davis, CA (US)

(21) Appl. No.: 17/790,594

(22) PCT Filed: Jan. 8, 2021
(86) PCT No.: PCT/US2021/012774

§ 371 Date: Jul. 1, 2022

Related U.S. Application Data
(60) Provisional application No. 62/959,657, filed on Jan. 10, 2020.

(51) Int. Cl. A61K 31/365; A61K 31/12; A61K 45/06

(52) U.S. Cl. CPC A61K 31/365; A61K 31/12; A61K 45/06

(57) ABSTRACT

Provided herein are methods of using andrographolide, oridonin, isoliquiritigenin, and derivatives thereof to upregulate β-defensin 3 expression in humans and other veterinary animals. Upregulated β-defensin 3 expression is useful in a number of contexts including the prevention of infections, promotion of mucosal health, treatment of wounds, and the treatment of respiratory conditions.

### CROSS-REFERENCES TO RELATED APPLICATIONS

This application claims the benefit of priority under 35 U.S.C. § 119(e) of U.S. Provisional Application No. 62/959,657 filed Jan. 10, 2020, the contents which is herein incorporated by reference in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Due to the rise of antimicrobial resistance amongst patients in the human and veterinary population, there has been a significant effort to develop novel therapies to target pathogens. Indeed, universities and private industry alike continue to invest significant efforts to identify novel antimicrobials that are suitable for pharmaceutical use. An additional strategy has focused on the exogenous expression (recombinant or synthetic) of antimicrobial peptides that are found naturally at host-microbe interfaces. However, these peptides typically require a costly complex biosynthesis that has served as a barrier to widespread development and commercialization.

An alternative strategy to synthesizing and preparing antimicrobial peptides would be to upregulate the endogenous expression of antimicrobial peptides that are found naturally at the host-microbe interface by providing an exogenous therapeutic that can upregulate the expression of these peptides. To date, no commercially available therapeutic exists to induce and increase expression of an endogenous antimicrobial peptide.

The present disclosure addresses this need and provides related advantages as well.

### SUMMARY

In some aspects, provided herein are methods of treating or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

In some aspects, provided herein are methods of promoting mucosal health or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

In some aspects, provided herein are methods of treating a wound comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

A method of treating a respiratory condition comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

### BRIEF DESCRIPTION OF THE DRAWINGS

FIG. 1 plots the relative DEFB103 (Human β-defensin 3) mRNA expression in telomerase immortalized human corneal epithelial cells when incubated with the indicated amount of andrographolide. The bars represent mean expression and the error bars represent standard deviation.

FIG. 2 plots the relative DEFB103 (Human β-defensin 3) mRNA expression in telomerase immortalized human corneal epithelial cells when incubated with the indicated amount of oridonin. The bars represent mean expression and the error bars represent standard deviation.

FIG. 3 plots the relative DEFB103 (Human β-defensin 3) mRNA expression in telomerase immortalized human corneal epithelial cells when incubated with the indicated amount of isoliquiritigenin. The bars represent mean expression and the error bars represent standard deviation.

FIG. 4 plots relative DEFB103 (Human β-defensin 3) mRNA expression in telomerase immortalized human corneal epithelial cells when incubated with the indicated amount of andrographolide for 24 hours. Bars represent mean values of 2-3 independent experiments normalized to media alone; error bars represent standard deviation. One-way ANOVA performed with Tukey's multiple comparisons test, different letters indicate significant differences (P<0.05).

FIG. 5 plots relative DEFB103 (Human β-defensin 3) mRNA expression in telomerase immortalized human corneal epithelial cells when incubated with the indicated amount of oridonin for 24 hours. Bars represent mean values of 2-3 independent experiments normalized to media alone; error bars represent standard deviation. One-way ANOVA performed with Tukey's multiple comparisons test, different letters indicate significant differences (P<0.05).

FIG. 6 plots relative DEFB103 (Human β-defensin 3) mRNA expression in telomerase immortalized human corneal epithelial cells when incubated with the indicated amount of isoliquiritigenin for 24 hours. Bars represent mean values of 2-3 independent experiments normalized to media alone; error bars represent standard deviation. One-way ANOVA performed with Tukey's multiple comparisons test, different letters indicate significant differences (P<0.05).

FIG. 7 plots the kinetics of DEFB103 (Human β-defensin 3) mRNA expression induction in human corneal epithelial (hTCEpi) cells. hTCEpi cells cultured to 80% confluence and treated in the presence of andrographolide (75 μM) for 2, 6, 12 or 24 hours prior to mRNA isolation. Vehicle controls included DMSO. Data points represent mean values of 2-3 independent experiments normalized to media alone, error bars represent standard deviation. Two-way ANOVA performed with Tukey's multiple comparisons test, indicating differences between treatment and vehicle control at specific time points.

FIG. 8 plots the kinetics of DEFB103 (Human β-defensin 3) mRNA expression induction in human corneal epithelial (hTCEpi) cells. hTCEpi cells cultured to 80% confluence and treated in the presence of oridonin (10 μM) for 2, 6, 12 or 24 hours prior to mRNA isolation. Vehicle controls included DMSO. Data points represent mean values of 2-3 independent experiments normalized to media alone, error bars represent standard deviation. Two-way ANOVA performed with Tukey's multiple comparisons test, indicating differences between treatment and vehicle control at specific time points.

FIG. 9 plots the kinetics of DEFB103 (Human β-defensin 3) mRNA expression induction in human corneal epithelial (hTCEpi) cells. hTCEpi cells cultured to 80% confluence and treated in the presence of isoliquiritigenin (24 μM) for 2, 6, 12 or 24 hours prior to mRNA isolation. Vehicle controls included EtOH. Data points represent mean values of 2-3 independent experiments normalized to media alone, error bars represent standard deviation. Two-way ANOVA performed with Tukey's multiple comparisons test, indicating differences between treatment and vehicle control at specific time points.

FIG. 10 plots the human β-defensin 3 (hBD3) peptide expression in hTCEpi cells treated with increasing amounts of andrographolide after 48 hours of incubation. Data are presented from single induction experiment and mean of two ELISA replicates.

### DETAILED DESCRIPTION OF THE INVENTION

### I. General

The field of medicine is facing a crisis of antibiotic resistance by microbes due to the excessive use of antibiotics and poor stewardship, therefore, there is a clinical need to develop alternative treatment strategies. Many species, including mammals, have genes that encode small peptides that possess broad-spectrum antimicrobial activity against bacteria, viruses, fungi, and some parasites. Importantly, there are limited reports of resistance to these endogenous antimicrobial peptides. Manufacturing these peptides for exogenous use has encountered significant obstacles, particularly in the cost and ability of manufacturing peptides with proper tertiary structure (disulfide bonding) of the molecule.

An alternative strategy to synthesizing these compounds is upregulating the endogenous expression of these peptides. Human β-defensin 3 (an endogenous antimicrobial peptide) exhibits broad-spectrum antimicrobial activity with direct killing of bacteria, fungus and inactivation of viruses, which is of particular relevance to human patients with infectious keratitis caused by Staphylococcus aureus or human herpes simplex virus 1 (HSV1). It is believed that β-defensin 3 in veterinary populations is also useful in the killing of bacteria, fungus and inactivation of viruses.

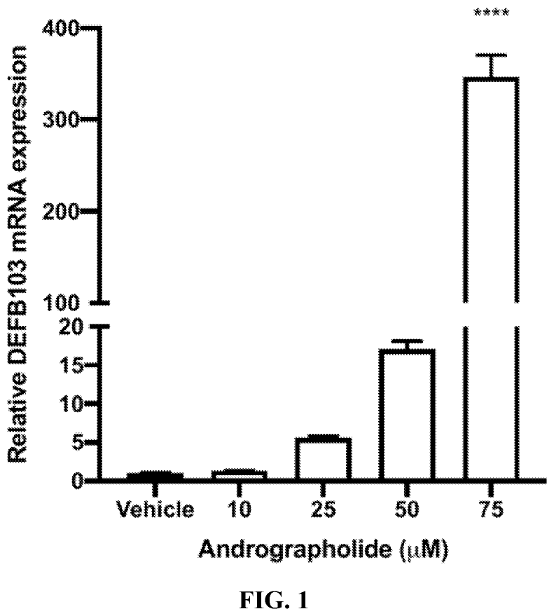

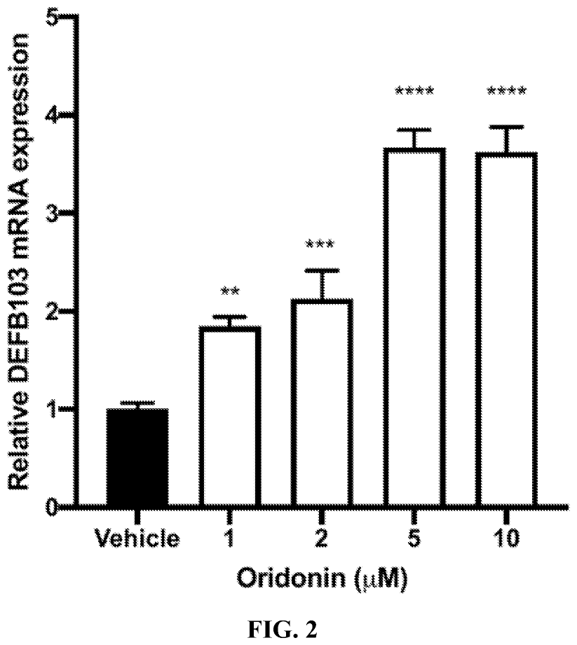

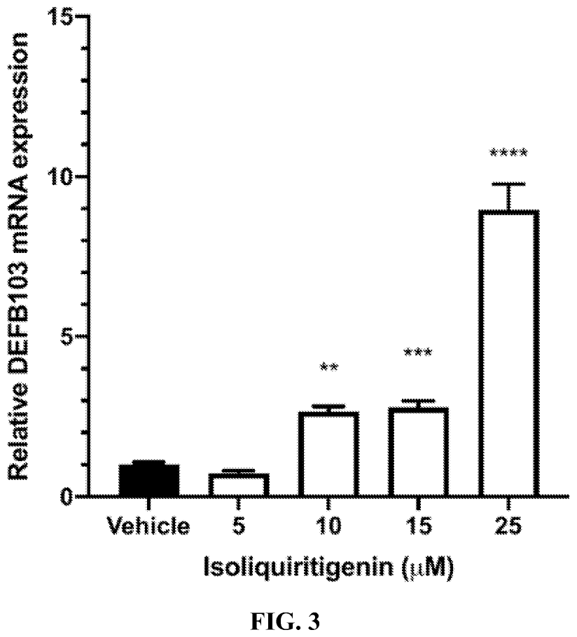

The current inventors have discovered the ability of andrographolide (labdane diterpenoid isolated from Andrographis paniculate), oridonin (ent-kaurane diterpenoid isolated from Rabdosia rubenscens), and isoliquiritigenin (simple chalcone-type flavonoid isolated from licorice root, Glycyrrhiza uralensis) to induce expression of human β-defensin 3 using immortalized human corneal epithelial cells (hTCEpi) in culture. Specifically, corneal epithelial cells treated with andrographolide, oridonin and isoliquiritigenin individually increased human β-defensin 3 mRNA expression 360-fold, 4-fold and 9-10-fold, respectively, over vehicle control alone (see, FIG. 1-3). Veterinary populations will also experience increased β-defensin 3 expression when administered andrographolide, oridonin, or isoliquiritigenin.

Based on these results, these herbal derived compounds and mimetics thereof are useful therapeutic options for patients with infections such as infectious keratitis or HSV1 infection. The compounds can be delivered in a variety of manners, which include a topical medication as well as impregnated into a device such as a contact lens or deployable implant for extended delivery. Other broad therapeutic applications are apparent for the treatment of chronic wounds (diabetic, chronic venous ulcers, and the like) as well as having application to promote mucosal health in conditions exemplified by but not limited to dry eye disease, oral pathologies related to dry mouth, oral thrush, trench mouth, and oral herpes infection. Also contemplated herein is delivery of these compounds with a nebulizer for treatment of severe/chronic/recurrent respiratory conditions.

### II. Detailed Description of Embodiments

A. Methods of Use

In some aspects, provided herein are methods of treating or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

In some aspects, provided herein are methods of promoting mucosal health or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

In some aspects, provided herein are methods of treating a wound comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

A method of treating a respiratory condition comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

Administering a compound described herein, optionally, with an additional therapeutic agent is useful in treating or preventing many diseases and disorders. These diseases and disorders include infectious diseases (e.g., caused by a virus, a bacterium, a fungus, a parasite, or any other infectious agent), promoting mucosal health, treating a wound, or treating a respiratory condition in a subject in need thereof.

The virus treated with the compositions of the present invention can be any known virus including, but not limited to, Filoviruses such as Ebola virus and Marburg virus, Crimean-Congo hemorrhagic fever virus, Human immunodeficiency virus (HIV), Herpes simplex, type 1, Herpes simplex, type 2, Human herpesvirus, type 8, Hepatitis A virus, Hepatitis B virus, Hepatitis C virus, Hepatitis D virus, Hepatitis E virus, Influenza virus, Parainfluenza virus, yellow fever virus, Varicella zoster virus, West Nile virus, dengue virus, Rabies virus, Measles virus, Mumps virus, poliovirus, Smallpox, Epstein-Barr virus, Human cytomegalovirus, Junin virus, Adenovirus, Orbivirus, Banna virus, Guanarito virus, Lassa virus, Rubella virus, JC virus, Machupo virus, Parvovirus B19, Hendra virus, Severe acute respiratory syndrome virus, Respiratory syncytial virus, BK virus, rhinovirus, coxsackievirus, Human papillomavirus, Norwalk virus, Human astrovirus, Human bocavirus, Human metapneumovirus, Rotavirus, Coltivirus, Sabii virus, and Nipah virus.

The bacterial infection treated with the compositions of the present invention can be any known bacterium including, but not limited to, Pseudomonas aeruginosa, Staphylococcus aureus, Streptococcus pneumoniae, tubercle bacilli, Bacillus anthracis, Escherichia coli, Salmonella typhimurium, Salmonella typhi, Salmonella thyphosa, Yersinia pestis, Vibrio cholerae, Clostridium perfringens, staphylococcus, Shigella, Klebsiella, Haemophilus influenzae, Pasteurella, Actinobacillus, Legionella, Bordetella pertussis, Francisella tularensis, Brucella, Vibrio parahaemolyticus, Neisseria gonorrhoeae, Neisseria meningitidis, Helicobacter pylori, Spirillum minus, Borrelia recurrentis, Borrelia burgdoferi, Clostridium tetani, Mycobacterium leprae, and Mycobacterium lepromatosis.

In some embodiments the bacterial infection is infectious keratitis. In some embodiments, the infectious keratitis is caused by Pseudomonas aeruginosa or Staphylococcus aureus.

The parasitic infection treated with the compositions of the present invention can be any known parasite including, but not limited to, Plasmodium, Schistosoma, Ascaris, Dracunculus Babesia, Toxoplasma, Eimeria, Isospora, Atoxoplasma, Cystoisospora, Hammondia, Besniotia, Sarcocystis, Frenkelia, Haemoproteus, Leucocytozoon, Theileria, Perkinsus, Nosema, Enterocytozoon, Encephalitozoon, E. intestinalis, Mrazekia, Amblyospora, Ameson, Glugea, and Pleistophora.

The fungal infection treated with the compositions of the present invention can be any known fungus including, but not limited to, Aspergillus, Blastomyces dermatitidis, Candida albicans, Coccidioides, Cryptococcus neoformans, Cryptococcus gattii, Histoplasma capsulatum, Mucoromycotina, Pneumocystis jirovecii, Sporothrix schenckii, and Exserohilum.

In some embodiments, the infection is on a mucosal surface. In some embodiments, the infection is a nasal infection, an oral infection, a respiratory infection, a vaginal infection, or corneal infection. In some embodiments, the infection is a corneal infection. In some embodiments, the infection is a respiratory tract infection, or an infection of the eye/cornea. In some embodiments, the infection is an infection of the eye/cornea.

In some embodiments, the infection is selected from the group consisting of infectious keratitis, oral thrush, trench mouth, and herpes simplex virus 1 infection.

In some embodiments, the compounds described herein are used in promoting mucosal health. An exemplary use is the prevention or treatment or dry eye disease.

In some embodiments, the compounds described herein are used in the treatment of a wound. In some embodiments, the wound is selected from the group consisting of diabetic foot wounds, burns, and radiation injury. In some embodiments the wound is a chronic wound. In some embodiments, the wound is caused by diabetes related complication or by chronic venous ulcers.

The terms subject, patient or individual are used herein interchangeably to include a human or animal. For example, the animal subject may be a mammal, a primate (e.g., a monkey), a livestock animal (e.g., a horse, a cow, a sheep, a pig, or a goat), a companion animal (e.g., a dog, a cat), a laboratory test animal (e.g., a mouse, a rat, a guinea pig, a bird), an animal of veterinary significance, or an animal of economic significance.

B. Compounds & Pharmaceutical Compositions of the Present Disclosure

Compounds useful for the presently disclosed methods include andrographolide, oridonin, isoliquiritigenin and derivatives thereof.

In some embodiments, andrographolide is used in the methods described herein. A person of skill in the art will recognize that andrographolide has the chemical structure shown below:

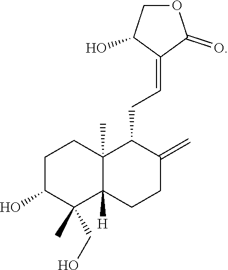 [SMILES: C=C1CC[C@@H]2[C@](C)(CO)[C@H](O)CC[C@@]2(C)[C@@H]1C/C=C1/C(=O)OC[C@H]1O]

In some embodiments a derivative of andrographolide is used in the methods described herein. In some embodiments derivatives of andrographolide include those described in U.S. Patent Application Publication No. US2018/0346438, the contents of which is hereby incorporated by reference for all purposes.

In some embodiments, oridonin is used in the methods described herein. A person of skill in the art will recognize that oridonin has the chemical structure shown below:

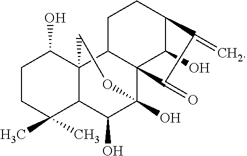

In some embodiments a derivative of oridonin is used in the methods described herein. In some embodiments derivatives of oridonin include those described in PCT Patent Application Publication No. WO2014/165841 or WO2017/062436, the contents of each are hereby incorporated by reference for all purposes.

In some embodiments, isoliquiritigenin is used in the methods described herein. A person of skill in the art will recognize that isoliquiritigenin has the chemical structure shown below:

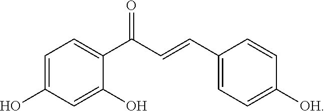 [SMILES: O=C(/C=C/c1ccc(O)cc1)c1ccc(O)cc1O]

In some embodiments a derivative of isoliquiritigenin is used in the methods described herein.

The compounds described herein are useful in the manufacture of a pharmaceutical composition or a medicament for preventing an infection, promoting mucosal health, treating a wound, or treating a respiratory condition in a subject in need thereof. In certain aspects, a pharmaceutical composition or medicament comprising one or more compounds described herein can be administered to a subject in combination with an additional therapeutic agent to prevent an infection, promote mucosal health, treat a wound, or treat a respiratory condition.

Pharmaceutical compositions or medicaments for use in the present invention can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Compounds and agents of the present invention and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof. In some embodiments, a compound described herein is dissolved in a liquid, for example, water. The most suitable route of administration for a compound or a therapeutic agent in any given case will depend, in part, on the type of therapeutic agent being used as well as the nature, severity, and optionally, the stage of the disease or condition being treating. In embodiments where the compound is administered in combination with an additional therapeutic agent, the administration of the compound and the therapeutic agent may be administered using the same or a different administration route. For example, in some embodiments, the compound may be administered orally, while the therapeutic agent may be administered subcutaneously.

The pharmaceutical compositions or medicaments of the present invention can include one or more compounds with one or more therapeutic agents or any pharmaceutically acceptable salts thereof, as an active ingredient and a pharmaceutically acceptable carrier and/or excipient or diluent. In some embodiments, the pharmaceutical compositions comprising a compound described herein and the pharmaceutical compositions comprising a therapeutic agent are prepared as separate medicaments. In some embodiments, the pharmaceutical compositions comprising a compound described herein and the pharmaceutical compositions comprising a therapeutic agent are prepared as a single medicament.

In embodiments where more than one compound described herein is used, the compounds can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

In certain embodiments, the pharmaceutical compositions or medicaments described herein are suitable for systemic administration. Systemic administration includes enteral administration (e.g., absorption of the compound through the gastrointestinal tract) or parenteral administration (e.g., injection, infusion, or implantation). In some embodiments, the pharmaceutical compositions or medicaments may be administered via a syringe or intravenously. In preferred embodiments, the pharmaceutical compositions or medicaments are injected subcutaneously.

In some embodiments, the present invention provides a pharmaceutical composition including a compound described herein, an antimicrobial agent, and a pharmaceutically acceptable excipient. In some embodiments, the compound described herein and the antimicrobial agent are separately prepared pharmaceutical compositions. In some embodiments, the pharmaceutically acceptable excipient includes a salt or a diluent.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethyleneglycol 6000 and titatium dioxide. Tablets may be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Typical formulations for topical administration include creams, ointments, sprays, lotions, and patches. Topical administration also includes topical ophthalmic delivery. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

Pharmaceutical compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art. In certain instances, the compositions may be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compounds described herein and the therapeutic agents can also be formulated in rectal compositions, for example, suppositories or retention enemas, for example, containing conventional suppository bases, for example, cocoa butter or other glycerides.

The compositions set forth herein can be formulated for parenteral administration by injection, for example by bolus injection. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the compound(s) can be in powder form for reconstitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the compound(s).

In some embodiments, the compounds are prepared with a polysaccharide such as chitosan or derivatives thereof (e.g., chitosan succinate, chitosan phthalate, etc.), pectin and derivatives thereof (e.g., amidated pectin, calcium pectinate, etc.), chondroitin and derivatives thereof (e.g., chondroitin sulfate), and alginates.

In some embodiments, the compositions further include a pharmaceutical surfactant. In other embodiments, the compositions further include a cryoprotectant. Non-limiting examples of cryoprotectants include glucose, sucrose, trehalose, lactose, sodium glutamate, PVP, cyclodextrin, 2-hydroxypropyl-13-cyclodextrin (HPI3CD) glycerol, maltose, mannitol, saccharose, and mixtures thereof.

C. Combination Therapy

In some embodiments, the compounds described herein are used as adjunctive therapies with an additional therapeutic agent. Additional therapeutic agents include, but are not limited to, antimicrobials (antimicrobial agent).

Antimicrobials are drugs, generally small molecules, that either kill microorganisms or inhibit their growth. Antimicrobials include antivirals, antibiotics, antifungals, and anti-parasitic agents, all of which are well known in the art. In some embodiments, the compounds described herein are useful for boosting a subject's expression of β-defensin 3 while the antimicrobial agent acts via a different mechanistic pathway. In certain instances, the patient overcomes the infection faster than using the antimicrobial agent alone.

Antivirals are drugs used in treating viral infections, and generally function by inhibiting a particular step in the virus life cycle. A person of skill in the art can appropriately determine which known antiviral medication to apply based on the virus infecting the individual. In some embodiments, antivirals include viral integrase strand transfer inhibitors. In some embodiments, antivirals include viral nucleoside reverse transcriptase inhibitors. In some embodiments, antivirals include viral neuraminidase inhibitors. In some embodiments, antivirals used to treat HIV/AIDS infections include, but are not limited to, tenofovir, lamivudine, emtricitabine, efavirenz, emtricitabine, rilpivirine, fosamprenavir, ritonavir, darunavir, atazanavir, dolutegravir, zidovudine, abacavir, and combinations thereof. In some embodiments, antivirals used to treat filovirus infections, such as Ebola virus, include, but are not limited to, favipiravir, brincidofovir, 3-deazaneplanocin A, amiodarone, dronedarone, verapamil, and combinations thereof. In some embodiments, antivirals used to treat influenza include, but are not limited to, laninamivir, oseltamivir, peramivir, zanamivir, and combinations thereof.

Antibiotics are generally small molecules that can either kill or inhibit the growth of bacteria. The most common antibiotics are those that target the bacterial ribosome. Because the ribosome is ubiquitous, most antibiotics can treat many different types of bacteria. The increase in antibiotic resistance, however, may require the use of one or more antibiotics to treat the infection. Antibiotic classes include, but are not limited to, penicillins, cephalosporins, macrolides, fluoroquinolones, sulfonamides, tetracyclines, and aminoglycosides. A person of skill in the art can appropriately determine which known antibiotic medication to apply based on the bacteria infecting the individual. Examples of antibiotics used to treat bacterial infections include, but are not limited to, penicillin, amoxicillin, doxycycline, azithromycin, erythromycin, roxithromycin, ciprofloxacin, flucloxacillin, phenoxymethylpenicillin, benzylpenicillin, ceftriaxone, metronidazole, cefaclor, cefadroxil, cephalexin, tetracycline, lymecycline, gentamicin, tobramycin, co-trimoxazole, S-649266, and combinations thereof.

Antifungals are drugs that can kill or prevent the growth of fungi. Targets of antifungal drugs include sterol biosynthesis, DNA biosynthesis, and β-glucan biosynthesis. A person of skill in the art can appropriately determine which known antifungal medication to apply based on the fungus infecting the individual. Examples of common antifungals include, but are not limited to, amphotericin B, nystatin, fluconazole, itraconazole, ketoconazole, naftifine, and combinations thereof.

Antiparasitic agents are drugs that can kill or prevent the growth of the parasite. The most common parasites infecting individuals are helminthes and parasitic protozoa, each of which are treated with different types of antiparasitic agents. A person of skill in the art can determine the most appropriate antiparasitic agent to apply based parasite being treated. Antiparasitic agents include, but are not limited to, antihelminthic agents such as mebendazole, pyrantel pamoate, thiabendazole, diethylcarbamazine, niclosamide, praziquantel, albendazole, and combinations thereof. Antiparasitic agents also include, but are not limited to, antiprotozoal agents such as melarsoprol, eflornithine, metronidazole, tinidazole, miltefosine, and combinations thereof. A particularly prevalent disease caused by a parasitic infection is malaria. Antiparasitic agents further include, but are not limited to, antimalarial agents such as rufigallol, quinine and related agents, chloroquine, amodiaquine, pyrimethamine, proguanil, sulfonamides, mefloquine, atovaquone, primaquine, artemisinin and derivatives, halofantrine, doxycycline, clindamycin, and combinations thereof.

D. Methods of Administration & Dosing

Pharmaceutical compositions or medicaments comprising a compound described herein can be administered to a subject at a therapeutically effective dose, optionally, in combination with an effective amount of an additional therapeutic agent. In some embodiments, the pharmaceutical composition or medicament comprising a compound described herein is administered to a subject in an amount sufficient to elicit an effective therapeutic response in the subject. In some embodiments, the pharmaceutical composition or medicament comprising a compound described herein can be administered to a subject at a therapeutically effective dose in combination with an effective amount of a therapeutic agent to elicit improved expression of β-defensin 3.

The compounds described herein and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof.

The combination therapy described herein includes simultaneous administration as well as sequential administration. In some embodiments, the pharmaceutical composition or medicament comprising a compound described herein is administered in a different time regiment than the therapeutic agent. As a non-limiting example, the pharmaceutical composition or medicament may be administered daily, while the additional therapeutic agent may be administered weekly, or the pharmaceutical composition or medicament may be administered weekly, while the therapeutic agent is administered daily.

The pharmaceutical composition or medicament comprising a compound described herein may be administered on a routine schedule (e.g., hourly, daily, every 3 days, weekly, monthly, yearly) or according to a cyclic schedule (e.g., 1 week of daily administration, 2 consecutive weeks without administration, or 3 consecutive weeks of daily administration, 3 consecutive weeks without administration, or 4 consecutive weeks of daily administration, 5 consecutive weeks without administration, and repeating the cycles as necessary).

In particular embodiments, one or more compounds described herein are administered in combination with an antimicrobial agent.

The dosage of compounds administered is dependent on the subject's body weight, age, individual condition, and/or on the form of administration. The size of the dose will also be determined by the existence, nature, and extent of any adverse effects that accompany the administration of a particular compound in a particular subject. Typically, a dosage of the active compounds is a dosage that is sufficient to achieve the desired effect. Optimal dosing schedules can be calculated from measurements of compound accumulation in the body of a subject. In general, dosage may be given once or more daily, weekly, or monthly. Persons of ordinary skill in the art can easily determine optimum dosages, dosing methodologies, and repetition rates.

The effective amount of a therapeutic agent administered with a compound described herein may be provided at the dosages generally recommended for each therapeutic agent used. In some instances, it may be necessary to increase or decrease the dosage levels to achieve the desired effect. For instance, some additional therapeutic agents when administered with compounds described herein may be effective at a lower dose. Optimal dosing schedules can be determined from measurements of compound accumulation in the body of a subject. Persons of ordinary skill in the art can easily determine optimum dosages, dosing methodologies, and repetition rates to optimize the effect of each therapeutic agent.

In some embodiments, a unit dosage for oral administration of a compound described herein to a subject (e.g., a human or other mammal) of about 50 to about 70 kg may contain between about 1 and about 500 mg, about 5 and about 500 mg, about 5 and about 250 mg, about 25 to about 250 mg, about 100 and about 1000 mg, about 200 and about 2000 mg, about 500 and about 5000 mg, or about 1000 and about 2000 mg of the compound(s). In particular embodiments, a unit dosage for oral administration of a compound described herein to a subject (e.g., human or other mammal) of about 50 to about 70 kg may contain about 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, 1000 mg, 1250 mg, 1500 mg, 2000 mg, 2500 mg, 3000 mg, or more of the compound(s).

In some embodiments, a unit dosage for topical administration of a compound described herein to a subject (e.g., a human or other mammal) may contain between about 1 and about 400 mg, about 5 and about 400 mg, about 5 and about 200 mg, about 25 to about 200 mg, about 100 and about 1000 mg. In particular embodiments, a unit dosage for topical administration of a compound described herein to a subject (e.g., human or other mammal) may contain about 5 mg, 10 mg, 15 mg, 20 mg, 25 mg, 30 mg, 35 mg, 40 mg, 45 mg, 50 mg, 75 mg, 100 mg, 200 mg, 300 mg, 400 mg, 500 mg, 600 mg, 700 mg, 800 mg, 900 mg, or 1000 mg or more of the compound(s).

In some embodiments, a unit dosage for subcutaneous administration of a compound described herein to a subject (e.g., human or other mammal) of about 50 to about 70 kg may contain between about 0.1 and about 100 mg, about 0.5 and about 100 mg, about 0.5 and about 50 mg, about 0.5 and about 25 mg, about 0.5 and about 10 mg, about 0.25 to about 50 mg, about 0.25 to about 25 mg, about 0.1 to about 50 mg, about 0.1 to about 25 mg, or about 0.1 to about 10 mg of the compound(s). In particular embodiments, a unit dosage for subcutaneous administration of a compound described herein to a subject (e.g., human or other mammal) of about 50 to about 70 kg may contain about 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 15 mg, 20 mg, 25 mg, 30 mg, 35 mg, 40 mg, 45 mg, 50 mg, 75 mg, 100 mg, or more of the compound(s).

When one or more of the compositions is to be administered to a mammal, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, a pharmaceutical composition or medicament of the present invention is administered orally, e.g., in a dose in the range of from about 1 to about 1000 micrograms (μg) of compound per kg of subject body weight, from about 1 to about 500 μg/kg body weight, from about 10 to about 1000 μg/kg body weight, from about 10 to about 500 μg/kg body weight, from about 50 to about 1000 μg/kg body weight, from about 50 to about 500 μg/kg body weight, from about 100 to about 1000 μg/kg body weight, or from about 100 to about 500 μg/kg body weight. In particular embodiments, the dose is about 10, 25, 50, 75, 100, 200, 300, 400, 500, 600, 700, 800, 900, or 1000 μg/kg body weight. The dose can be administered once per day or divided into sub-doses and administered in multiple doses, e.g., twice, three times, or four times per day. However, as will be appreciated by a skilled artisan, oral compositions described herein may be administered in different amounts and at different times.

In some embodiments, a pharmaceutical composition or medicament of the present invention is administered subcutaneously, e.g., in a dose in the range of from about 1 to about 500 micrograms (μg) of compound per kg of subject body weight, from about 1 to about 200 μg/kg body weight, from about 1 to about 100 μg/kg body weight, from about 10 to about 500 μg/kg body weight, from about 10 to about 200 μg/kg body weight, from about 10 to about 100 μg/kg body weight, or from about 10 to about 80 μg/kg body weight. In particular embodiments, the dose is about 1, 5, 10, 15, 20, 25, 30, 35, 40, 45, 50, 55, 60, 65, 70, 75, 80, 85, 90, 95, 100, 200, 300, 400, or 500 μg/kg body weight. The dose can be administered once per day or divided into sub-doses and administered in multiple doses, e.g., twice, three times, or four times per day. However, as will be appreciated by a skilled artisan, subcutaneous compositions described herein may be administered in different amounts and at different times.

In some embodiments, the compounds are administered for about 1 to about 31 days, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the compounds are administered for at least 1 day. In other embodiments, the compounds are administered for one or more weeks, e.g., 1, 2, 3, 4, 5, 6, 7, 8, 9, 10, 11, 12, 13, 14, or more weeks. In yet other embodiments, the compounds are administered for one or more months, e.g., 1, 2, 3, 4, 5, 6, 7, 8, 9, 10, 11, 12, or more months.

To achieve the desired therapeutic effect, compounds may be administered for multiple days at the therapeutically effective daily dose. Thus, therapeutically effective administration of compounds to treat a pertinent condition or disease described herein in a subject requires periodic (e.g., daily or twice daily) administration that continues for a period ranging from three days to two weeks or longer. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every day, every other day, or, if higher dose ranges are employed and tolerated by the subject, twice a week.

Optimum dosages, toxicity, and therapeutic efficacy of such compounds may vary depending on the relative potency of individual compounds and can be determined by standard pharmaceutical procedures in experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While compounds that exhibit toxic side-effects can be used, care should be taken to design a delivery system that targets such compounds to the affected site to minimize potential damage to normal cells and, thereby, reduce side-effects.

The data obtained from, for example, animal studies can be used to formulate a dosage range for use in humans or other mammals. The dosage of such compounds lies preferably within a range of circulating concentrations that include the ED₅₀ with little or no toxicity. The dosage can vary within this range depending upon the dosage form employed and the route of administration.

A dose can be formulated in animal models to achieve a concentration range that includes the IC₅₀ (the concentration of the agent that achieves a half-maximal inhibition of symptoms) as determined in cell culture. Such information can be used to more accurately determine useful doses in humans and other mammals. Levels in stool or an enteric tissue sample can be measured, for example, by high performance liquid chromatography (HPLC). In general, the dose equivalent of compounds is from about 1 ng/kg to about 500 mg/kg for a typical subject.

The dosage of a pharmaceutical composition or medicament of the present invention can be monitored and adjusted throughout treatment, depending on severity of symptoms, frequency of recurrence, and/or the physiological response to the therapeutic regimen. Those of skill in the art commonly engage in such adjustments in therapeutic regimens.

Single or multiple administrations of the pharmaceutical compositions or medicaments can be administered depending on the dosage and frequency as required and tolerated by the patient. In any event, the composition or medicament should provide a sufficient quantity of the compounds of the invention to effectively treat the patient. Generally, the dose is sufficient to enhance the expression of β-defensin 3 without producing unacceptable toxicity or side-effects to the patient.

### III. EXAMPLES

### Example 1: Upregulation of Human β-Defensin 3

Measuring mRNA Expression Levels

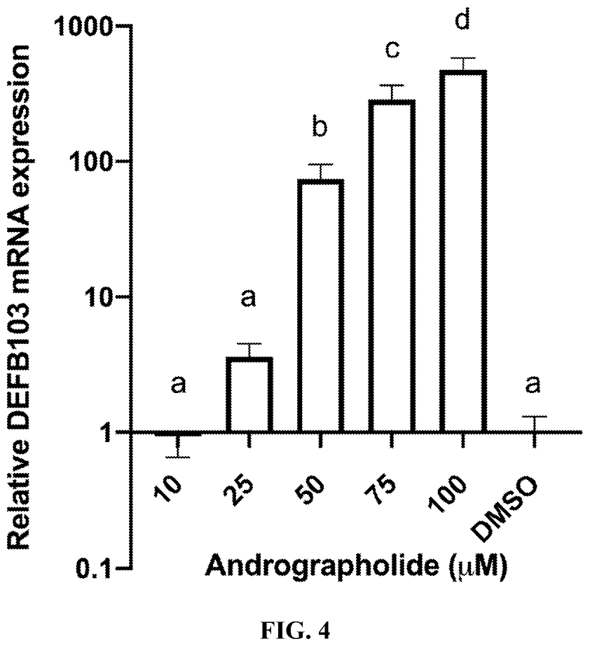

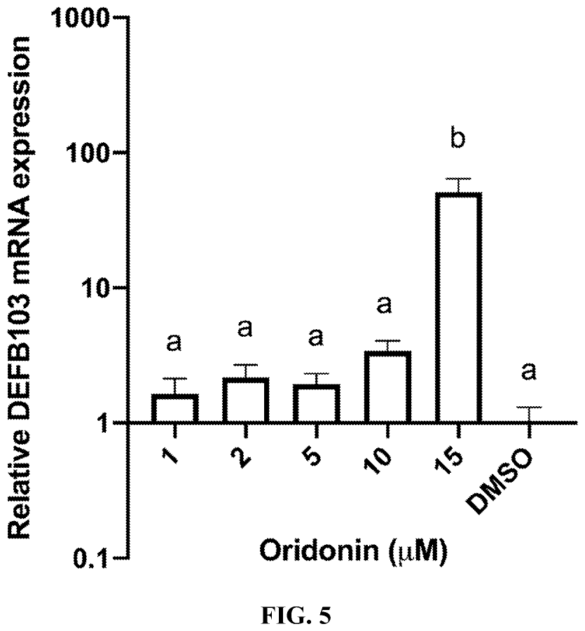

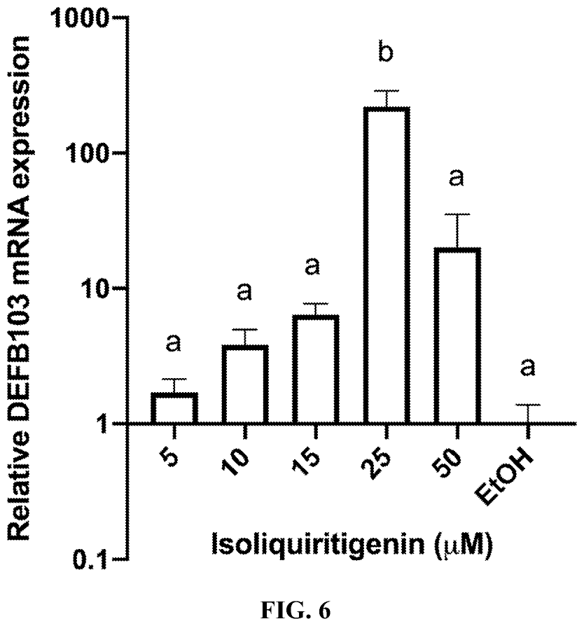

Telomerase immortalized human corneal epithelial cells (hTCEpi cells, passage 40-50) were grown in 6-well plates to 80-90% confluence. Cells were treated with andrographolide, oridonin, isoliquiritigenin (Selleck Chem) individually for 24 hours, at concentrations previously determined to have no effect on cellular viability based on cell morphology, MTT assay and RNA yield. Vehicle control type was based on manufacturers' diluent recommendation and concentration was equivalent to the diluent concentration in the highest treatment (andrographolide and oridonin: DMSO, isoliquiritigenin: ethanol). After 24 hours, media was aspirated from each well and washed with sterile PBS. Total RNA was extracted (GeneJet) and quantified (NanoDrop). Approximately 50 ng of total RNA from each sample was reverse transcribed and quantitative PCR was performed using aptamers for DEFB103 (ThermoFisher), and GAPDH specific aptamers were used as a control. Expression of DEFB103 was calculated using the 2\hat{ }-ddCt method. A one-way ANOVA was performed to determine statistical significance with a post-hoc test to determine individual differences. Results are shown in FIG. 1-FIG. 3. Replicate experiments following the same protocol are shown in FIG. 4-6.

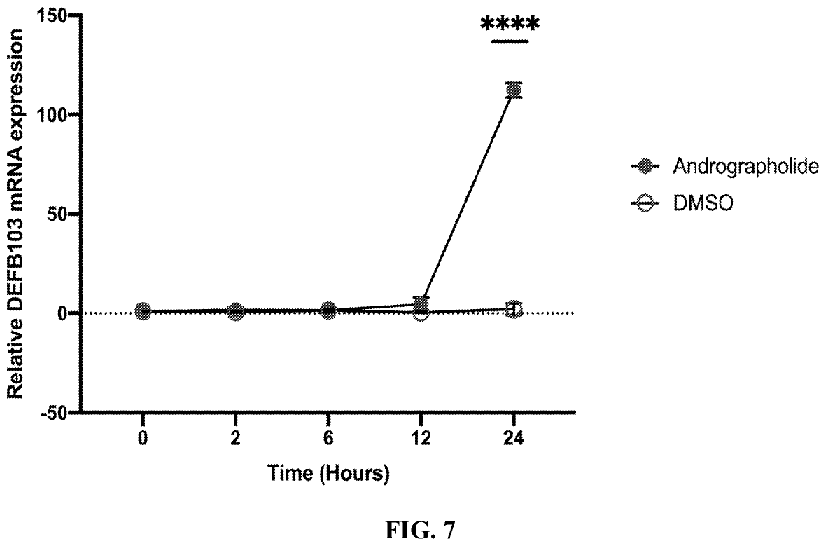

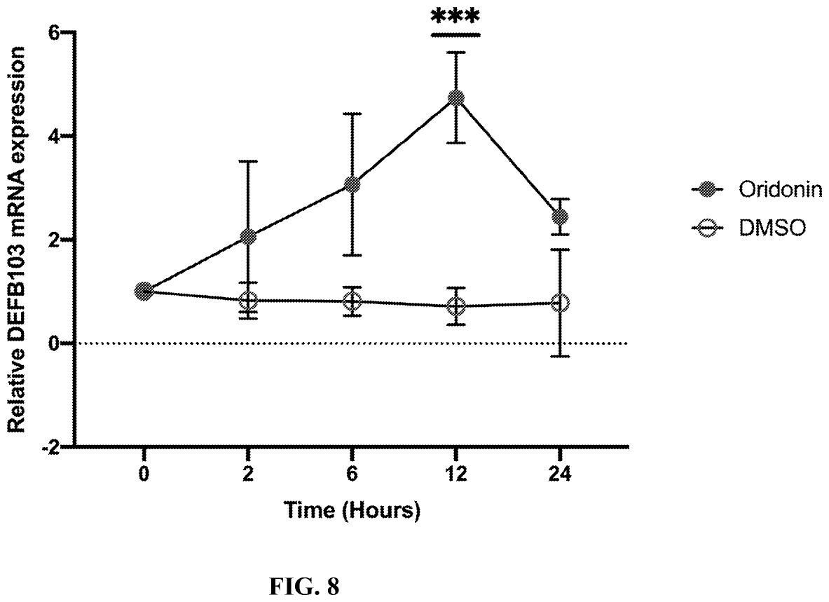

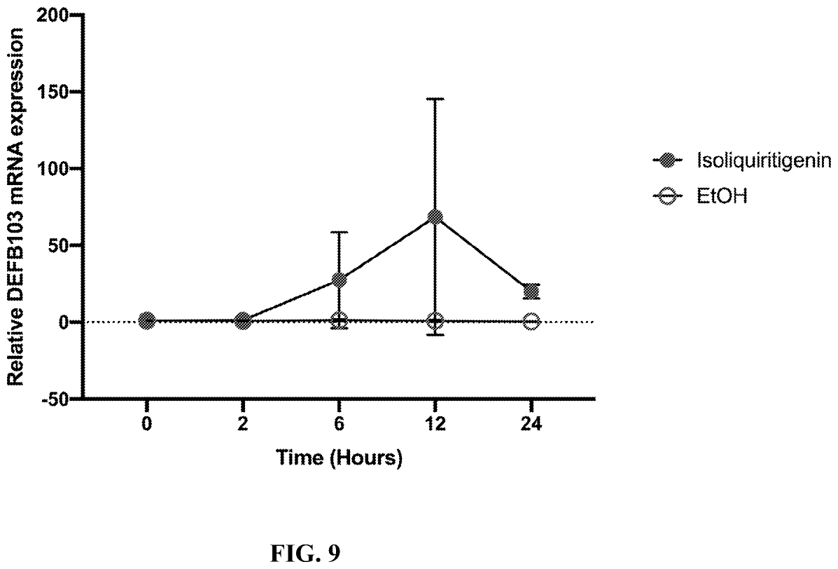

Separate time course experiments were performed as described above, where data points were taken at 2, 6, 12, and 24 hours. Results from these experiments shows that kinetics of human β-defensin 3 mRNA expression induction is human corneal epithelial (hTCEpi) cells is compound dependent. Results of these experiments are shown in FIG. 7-FIG. 9.

### Measuring Protein Expression Levels

hTCEpi cells were seeded into a 6-well plate coated with FNC material and allowed to grow to 80% confluence. The cells were subsequently treated with increasing concentrations of andrographolide (10, 25, 50, 75, 100 μM), oridonin (1, 2, 5, 10, 15 μM), isoliquiritigenin (5, 10, 15, 25, 50 μM) for 48 hours, and a negative control (vehicle: DMSO or EtOH). After treatment, the supernatant was collected and 100 μl was used to quantify hBD3 peptide concentration (Defensin 3, beta (human) ELISA Kit, Phoenix Pharmaceuticals Inc) and performed in duplicate for each treatment. An hBD3 peptide standard curve was generated from the ELISA kit reagents. The concentration of each experimental well was determined from the linear regression equation generated from the absorbance values of the hBD3 standards. However, hBD3 peptide concentrations were below the limit of detection at baseline and after treatment with increasing concentrations of oridonin and isoliquiritigenin. It is important to note that the majority of the AMP research fails to detect peptide concentrations from both in vitro and in vivo experiments despite observing upregulation at the mRNA level and the ability to augment antimicrobial activity of cells in either context.

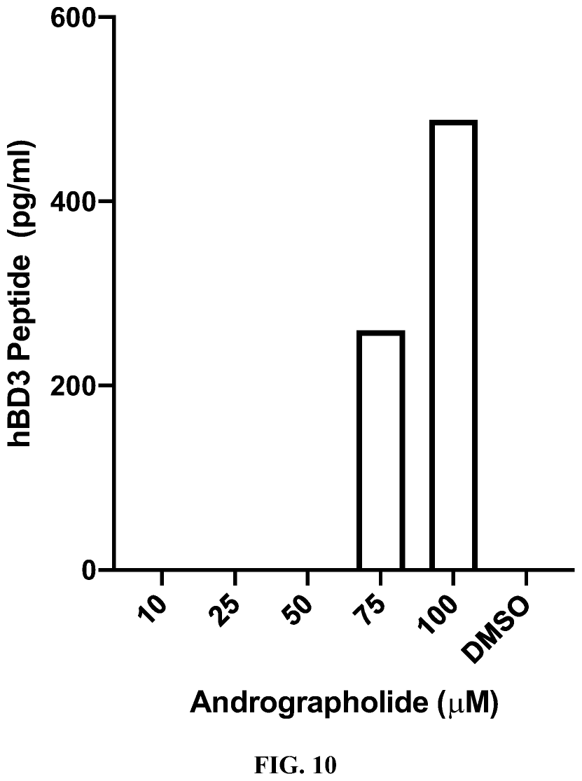

FIG. 10 shows that human β-defensin 3 (hBD3) peptide expression is markedly upregulated in a dose-dependent manner in hTCEpi cells treated with andrographolide. ELISA results from oridonin and isoliquiritigenin were below the limit of detection using the commercial ELISA kit.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

2. The method of claim 1, wherein the infection is a microbial infection.

3. The method of claim 1, wherein the infection is a viral, bacterial, or parasitic infection.

4. The method of any one of claims 1 to 3, wherein the infection is on a mucosal surface.

5. The method of any one of claims 1 to 3, wherein the infection is a nasal infection, an oral infection, a respiratory infection, a vaginal infection, or corneal infection.

6. The method any one of claims 1 to 3, wherein the infection is a respiratory tract infection, or an infection of the eye/cornea.

7. The method of any one of claims 1 to 3, wherein the infection is an infection of the eye/cornea.

8. The method of claim 1 or any one of claims 4 to 6, wherein the infection is selected from the group consisting of infectious keratitis, oral thrush, trench mouth, and herpes simplex virus 1 infection.

9. The method of claim 1 or any one of claims 4 to 6, wherein the infection is infectious keratitis.

10. A method of promoting mucosal health or preventing an infection comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

11. The method of claim 10, wherein the subject suffers from dry eyes.

12. A method of treating a wound comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

13. The method of claim 12, wherein the wound is selected from the group consisting of diabetic foot wounds, burns, and radiation injury.

14. The method of claim 12, wherein the wound is a chronic wound.

15. The method of claim 12, wherein the chronic wound is caused by diabetes related complication or by chronic venous ulcers.

16. A method of treating a respiratory condition comprising administering to a subject in need thereof an effective amount of a compound selected from the group consisting of andrographolide, oridonin, isoliquiritigenin, a derivative of andrographolide, a derivative of oridonin, and a derivative of isoliquiritigenin.

17. The method of any one of claims 1 to 16, wherein the compound is andrographolide.

18. The method of any one of claims 1 to 16, wherein the compound is a derivative of andrographolide.

19. The method of claim 18, wherein the derivative of andrographolide has the Formula 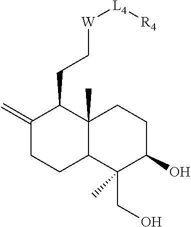
wherein,
W is O, N(R₅) or a ring 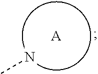
R₅ is selected from H, or from a C₁₋₃ alkyl optionally substituted by halogen, OH, NH₂, COOH, NHMe, or N(Me)₂;
L₄ is selected from a single bond and —(CRR)₁₋₃—;
R₄ is selected from H, COOH, or is independently selected from the group consisting of NH₂, C₁₋₆ alkyl, C₁₋₆ heteroalkyl, a 5-10 membered aryl, and a 5-10 membered heteroaryl, wherein the aryl or heteroaryl group are optionally substituted with 1, 2, or 3 R or R′;
the ring 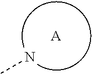
is selected from a 4-10 membered ring which is optionally substituted with 1, 2, or 3 R′;
R is independently selected from F, Cl, Br, I, OH, NH₂, CN, C(═O)OH, or from the group consisting of a C₁₋₆ alkyl, a C₁₋₆ heteroalkyl, a -L-C₃₆ cycloalkyl, and a -L-3-6 membered heterocycloalkyl, which is optionally substituted with 1, 2 or 3 R′;
L is selected from a single bond, —O—, —S—, —C(═O)NH—, —NH—, —C(═O)O—, —C(═O)—, —C(═S)—, —S(═O)—, —S(═O)₂;
R′ is independently selected from halogen, CN, OH, N(CH₃)₂, NH(CH₃), NH₂, CHO, C(═O)OH, C(═O)NH₂, S(═O)NH₂, S(═O)₂NH₂, trihalomethyl, dihalomethyl, monohalomethyl, aminomethyl, hydroxymethyl, methyl, methoxy, formyl, methoxycarbonyl, Boc, methylsulfonyl, methylsulfinyl, ethyl, n-propyl, isopropyl, C₃₋₆ membered cycloalkyl, and 3-6 membered heterocycloalkyl.

20. The method of any one of claims 1 to 16, wherein the compound is oridonin.

21. The method of any one of claims 1 to 16, wherein the compound is a derivative of oridonin.

22. The method of any one of claims 1 to 16, wherein the compound is isoliquiritigenin.

23. The method of any one of claims 1 to 16, wherein the compound is a derivative of isoliquiritigenin.

24. The method of any one of claims 1 to 23, wherein the compound is administered topically.

25. The method of any one of claims 1 to 23, wherein the compound is nebulized for nasal or respiratory administration.

26. A medical device comprising a compound selected from the group consisting of andrographolide, oridonin, isoliquiritigenin, a derivative of andrographolide, a derivative of oridonin, and a derivative of isoliquiritigenin

27. The method of claim 26, wherein the device is a contact lens.

28. The method of claim 26, wherein the device is implanted into a subject in need thereof.

29. The method of claim 26, wherein the device comprises a thin topcoat polymer for controlled release of the compound is implanted into a subject in need thereof.
